(19) United States
(12) Patent Application Publication
(10) Pub. No.: US 2009/0187253 A1
(43) Pub. Date: Jul. 23, 2009

(54) METHOD OF MAKING A COATED MEDICAL BONE IMPLANT AND A MEDICAL BONE IMPLANT MADE THEREOF

(75) Inventors: Maria Astrand, Storvreta (SE); Axel Genvad, Bandhagen (SE)

Correspondence Address: DRINKER BIDDLE & REATH (DC), 1500 K STREET, N.W., SUITE 1100, WASHINGTON, DC, 20005-1209, US

(73) Assignee: Sandvik Intellectual Property AB, Sandviken (SE)

(21) Appl. No.: 12/355,355

(22) Filed: Jan. 16, 2009

(30) Foreign Application Priority Data
Jan. 18, 2008 (SE) 0800127-3

(51) Int. Cl. A61F 2/28; C23C 14/36

(52) U.S. Cl. 623/23.6; 204/192.15

(57) ABSTRACT

The present invention relates to a method of making a coated medical bone implant comprising the step of providing a substrate and then onto said substrate depositing a bioactive crystalline TiO₂ coating using PVD (Physical Vapor Deposition) technique at a temperature of more than about 50° C. but less than about 800° C.

Coated implants obtained by the method according to the invention display an enhanced biomimetic response.

### CROSS-REFERENCE TO PRIOR APPLICATION

This application claims priority to Sweden Application No. 0800127-3 filed Jan. 18, 2008, which is incorporated by reference herein.

### BACKGROUND OF THE INVENTION

The present invention relates to a method of making a coated medical bone implant with a bioactive crystalline TiO₂ coating, where the TiO₂ coating has been deposited using PVD technique. Coated bone implants obtained by the method according to the invention display an enhanced biomimetic response.

Applying coatings to medical bone implants such as hip joints etc. is well known in the art. Coatings are applied for different reasons, e.g., increased wear resistance, improved biocompatibility and/or bioactivity.

Titanium and titanium alloys are well recognized materials for dental and orthopedic implants due to their good biocompatibility. On bone implants made of titanium, a thin surface layer of native titanium dioxide is immediately formed when exposed to air. Such layers have an amorphous crystal structure and are responsible for the good biocompatibility. By biocompatible is meant that the implant is inert and that it does not cause any toxicity or negative side effects to the tissue.

For some implant surfaces, i.e., those that are meant to bond with bone tissue, it is of high importance to have good bioactivity. By bioactive is meant that the material is capable of biochemically bonding to the natural tissue. This can only be achieved by having a more crystalline titanium oxide, i.e., an oxide with larger crystal grains. To obtain a more crystalline structure the oxidization can be forced by e.g., performing the oxidation of the Ti surface at an increased temperature. TiO₂ can also be deposited onto the surface of the implant as an additional coating/layer. This can for example be done by anodization, plasma spraying etc.

For implants such as dental and orthopedic implants, it is in some cases very important that the implant is bonded to the natural bone tissue as fast as possible, i.e., that it is osseointegrated. This means that hydroxyapatite needs to be formed rapidly on the implant surface. This, in turn, requires that the surface of the implant is both biocompatible and bioactive.

Vapor deposition processes such as CVD (Chemical Vapor Deposition) and PVD (Physical Vapor Deposition) are common techniques for coating semiconductors, optical surfaces, cutting tools etc. These techniques have also been used to coat implant surfaces where an increased wear resistance is wanted e.g., the contact zones in a hip joint, or as a corrosion barrier.

US 2003/0175444 A1 describes a method of coating artificial organs of organic and inorganic materials, such as vascular stents, artificial heart valves etc., with a plasma immersion ion implantation method (PIII). TiO₂ coatings, with a coating thickness of 0.05-5 μm, are deposited in a vacuum chamber by means of a metal arc plasma source which creates titanium plasma in the presence of oxygen gas or plasma. The artificial organs that are provided with the TiO₂ coating are suitable for implanting into human bodies and contacting blood. The artificial organs show improved blood compatibility i.e., improved anticoagulation properties. US 2003/0175444 A1 does not mention implants osseointegration i.e., implanting into bone.

WO 03/070288 describes a multilayered coating for implants comprising a first dense layer and a second bioactive layer. The first layer can be an oxide, nitride boride, carbide or mixtures thereof, preferably a nitride. The second layer is an apatite layer. The first layer will function as a corrosion barrier whereas the second layer is bioactive. The first layer can be deposited by PVD or CVD technique, oxides are preferably deposited using CVD.

However, very few attempts have been done to use vapor deposition techniques to deposit bioactive coatings, i.e., coatings that will create biochemical bonds to bone tissue.

The bioactivity of PVD deposited TiO2 has been evaluated in “Plasma-controlled nanocrystallinity and phase composition of TiO₂: a smart way to enhance biomimetic response”, J. Biomed. Mater. Res. Part. ADOI 10.1002 (2007) 453-464. The TiO₂ coatings have been deposited at room temperature, without preheating, by reactive DC magnetron technique. The bioactivity was evaluated by measuring the hydroxyapatite growth after immersion in simulated body fluid (SBF). The effect on bioactivity of the two different TiO₂ phases, rutile and anatase, were investigated.

However, there are some disadvantages with coatings deposited at room temperature of which one is related to the presence of water vapor. It is very important that all water is evaporated from the substrate surface prior to deposition. If water is still present on the surface, the adhesion of the coating will be compromised which would be a big disadvantage, especially on a medical implant that is aimed to stay in the body for a long time.

### OBJECTS AND SUMMARY OF THE INVENTION

It is an object of the present invention to provide a method of making a medical bone implant having a bioactive crystalline TiO₂ coating resulting in improved biomimetic response.

It is another object of the present invention to provide a method which gives coatings with good adhesion to the substrate.

In one aspect of the invention, there is provided a method of making a coated medical bone implant, comprising the steps of: providing a substrate, and onto said substrate depositing a bioactive crystalline TiO₂ coating using PVD technique at a deposition temperature of more than about 50° C. but less than about 800° C.

In another aspect of the invention, there is provided a coated medical bone implant comprising a substrate and a coating wherein the coating comprises a bioactive crystalline TiO₂ PVD coating.

### DETAILED DESCRIPTION OF THE PREFERRED EMBODIMENTS

The present invention relates to a method of making a coated medical bone implant comprising the step of: providing a substrate and onto said substrate depositing a bioactive crystalline TiO₂ coating by using PVD (Physical Vapor Deposition) technique at a temperature of more than about 50° C. but less than about 800° C.

By bone implant is meant any medical implant comprising at least one surface that is aimed for osseointegration, i.e., that the implant bonds to natural bone tissue being either human or animal. Examples of such implants are orthopedic prostheses for the hip, knee, ankle, shoulder, elbow and spine as well as dental implants. By bone implants are also meant devices for attachment of implants such as screws, nails etc.

PVD techniques suitable for the present invention are any PVD technique known in the art. Preferably any one of cathodic arc evaporation, magnetron sputtering or e-beam evaporation, most preferably cathodic arc evaporation, is used.

Prior to placing the substrates in the PVD chamber, the substrates are mounted on a rotating substrate holder. For complex geometries, a 3-fold rotation is preferably used.

The PVD process comprises several steps. First, the pressure is reduced in the chamber by removing the air by pumping, then the substrates are preheated to a suitable temperature after which the substrates are ion-etched, preferably using Ar ions, to remove any surface contaminants. Thereafter, the substrates are coated with titanium oxide using one or more pure Ti sources and by introducing oxygen into the deposition chamber. Evaporation of Ti atoms and/or ions can be performed using different techniques. For example, in cathodic arc evaporation, the source material is vaporized by melting a spot on the source using an arc, whereas in magnetron sputtering the Ti ions are vaporized by ion bombardment of the source surface. In e-beam evaporation the Ti is melted and vaporized using an electron beam. The degree of ionization of the Ti atoms depends on the chosen technique, however the Ti ions in the plasma will react with the oxygen, resulting in a film of TiO₂.

The deposition time varies depending on the chosen PVD technique and the wanted coating thickness.

The coating thickness for the deposited TiO₂ coating according to the present invention can be more than about 3 nm, preferably more than about 5 nm and most preferably more than about 10 nm, but less than about 5000 nm, preferably less than about 1000 nm, and most preferably less than about 500 nm.

The coating process according to the present invention is performed at a temperature of more than about 50° C., preferably more than about 70° C., and most preferably more than about 100° C., but less than about 800° C., preferably less than about 700° C., and most preferably less than about 550° C.

In one embodiment, the PVD technique used is cathodic arc evaporation. Then, the substrate bias is suitably from about 0 to about −500 V, preferably from about −5 to to about −300 V, and most preferably from about −10 to about −200 V. The arc current suitably is from 50 to about 250 A, preferably from about 65 to about 240 A, and most preferably from about 80 to about 220 A. The reactive gas flow preferably is from about 50 to about 2000 sccm, and most preferably from about 200 to about 1500 sccm.

The bioactive crystalline TiO₂ coating according to the present invention can have any crystalline phase but are preferably rutile or anatase or a mixture thereof.

By crystalline TiO₂ is herein meant that the coating results in diffraction spots or rings when analyzed using Selected Area Electron Diffraction Transmission Electron Microscopy (SAED-TEM). A crystalline TiO₂ coating according to the present invention can, if the measurements are performed by using X-ray Diffraction (XRD), appear to be amorphous. This can either be due to the low thickness and/or the small crystallites in the coating. Hence TEM analysis is, or can be, necessary to detect the crystallinity of the coating. In one embodiment of the present invention, the bioactive crystalline TiO2 coating has a crystalline phase which is a mixture of rutile and anatase. The different phases are identified by measurements either by X-ray Diffraction (XRD) or Selected Area Electron Diffraction Transmission Electron Microscopy (SAED-TEM).

Although, the present invention relates to a TiO₂ coating some deviation from the exact stoichiometry can be present.

The stoichiometry of the crystallites is close to TiO₂, as analysed using TEM. However, the coating in its whole might consist of small crystallites of stoichiometric TiO₂ in an amorphous non-stoichiometric matrix and hence the overall composition of the coating might deviate from TiO₂ stoichiometry. Hence, high-resolution microscopy such as TEM is necessary to evaluate the stoichiometry of the crystallites in the coating.

The bioactive crystalline TiO₂ coating can also contain other elements but then at a level of a technical impurity.

In one embodiment of the present invention, the bioactive crystalline TiO₂ layer is the outermost layer i.e., there can be other coatings present at the substrate surface, under the bioactive crystalline TiO₂ layer.

The substrate material can be any material suitable for implants. Examples of such materials are titanium, titanium-alloys, cobalt, cobalt alloys, tool steel, stainless steel, cobalt, Co—Cr—Mo-alloys.

The invention is additionally illustrated in connection with the following examples, which are to be considered as illustrative of the present invention. It should be understood, however, that the invention is not limited to the specific details of the examples.

### EXAMPLE 1

Substrates in the form of metal plates, 20×20×1 mm, were coated with TiO₂ using a cathodic arc evaporation PVD process. Three different substrate materials were used: commercially pure Ti grade 2, TiAl6V4 and Stainless steel, medical grade AISI type 316L.

Prior to deposition, the substrates were ultrasonically cleaned in acetone for 10 minutes followed by 10 minutes in ethanol before they were dried in hot air.

The substrates were mounted on a 3-fold rotating table which then was placed inside the PVD chamber, in which 4 sources of pure Ti had been mounted. The substrates were then heated for a period of 50 minutes to the aimed deposition temperature, see Table 1 below, followed by 36.5 min of Ar etching to remove any surface contaminants. During deposition the flow rate of oxygen was 800 sccm. The substrate bias was −60 V, the arc source power was 5-6 kV and the arc current 150 A. The deposition time, the deposition temperature and the thickness of the TiO₂ layer is given in Table 1. The thickness of the coatings was measured with a scanning electron microscope (SEM). Also, the crystal structure of the coatings was analyzed by X-ray diffraction (XRD). All coatings showed a mixture of rutile and anatase crystal structure.

TABLE 1

| | | Deposition | Deposition | |
|---|---|---|---|---|
| | | temp. | time | Coating thickness |
| Sample | Substrate | (° C.) | (min) | (nm) |
| Invention 1 | Ti, grade 2 | 320 | 40 | 1450 |
| Invention 2 | Ti, grade 2 | 500 | 10 | 290 |
| Invention 3 | Ti, grade 2 | 200 | 10 | 370 |
| Invention 4 | TiAl6V4 | 320 | 10 | 350 |
| Invention 5 | Stainless | 320 | 10 | 350 |
| | steel | | | |
| Invention 6 | Ti, grade 2 | 320 | 1 | 50 |

### EXAMPLE 2

A substrate of commercially pure Ti grade 2, in the form of metal plates, 20×20×1 mm, was coated with TiO₂ using a magnetron sputtering PVD process.

The substrates were first ultrasonically cleaned, first 6 minutes in a basic solution, then the substrates were rinsed before ultrasonically cleaned in ethanol for 6 minutes. Finally the samples were rinsed and dried in pure nitrogen gas.

The substrates were mounted on a holder that moves in a circular orbit and at the same time rotates around its own axis which then was placed inside the PVD chamber, in which one solid Ti source had been mounted. The substrates were then heated for a period of 60 minutes to the aimed deposition temperature, followed by 6 min of Ar etching to remove any surface contaminants. The substrate bias was +150 V, the total pressure during deposition was 4.2 μbar and the ratio of Ar:O₂ was 30:70. The deposition temperature was 200° C.

TABLE 2

| | | | Coating thickness |
|---|---|---|---|
| | Substrate | Deposition time (min) | (nm) |
| Invention 7 | Ti, grade 2 | 40 | 170 |

The thickness of the coating was measured by a scanning electron microscope (SEM). Also, the crystal structure of the coating was analyzed by X-ray diffraction (XRD). The coating showed a mixture of rutile and anatase crystal structure as measured by XRD.

### EXAMPLE 3

To evaluate the bioactivity i.e., the hydroxyapatite (HA) forming ability of the TiO₂ coatings biomimetics was used where the surface is tested in a simulated body fluid (SBF).

The samples from Example 1 and 2 were tested as well as reference samples as shown in Table 3:

TABLE 3

| | Substrate | Method |
|---|---|---|
| Ref. 1 | Ti, grade 2 | Exposure to air (Native TiO₂) |
| Ref. 2 | Ti, grade 2 | Thermal oxidation |
| Ref. 3 | TiAl6V4 | Thermal oxidation |

All samples were soaked in SBF. SBF is a fluid which has an ion composition and concentration similar to those of blood plasma. The SBF used in these tests were Dulbecco's phosphate buffered saline (PBS).

The samples were soaked in the SBF for a period of one week in 37° C. and then rinsed and dried. The growth of HA onto the TiO₂ coating surface was visually determined by a scanning electron microscope (SEM) and graded as good or poor. By “good” is herein meant that the HA layer is smooth and is covering the whole TiO₂ surface. By “poor” is meant that the HA growth does not cover the TiO₂ surface completely. The results are shown in Table 4.

TABLE 4

| | | | | HA |
|---|---|---|---|---|
| Sample | Substrate | Coating method | TiO₂ (nm) | growth |
| Invention 1 | Ti, grade 2 | Arc | 1450 | Good |
| Invention 2 | Ti, grade 2 | Arc | 290 | Good |
| Invention 3 | Ti, grade 2 | Arc | 370 | Good |
| Invention 4 | TiAl6V4 | Arc | 350 | Good |
| Invention 5 | Stainless | Arc | 350 | Good |
| | steel | | | |
| Invention 6 | Ti, grade 2 | Arc | 50 | Good |
| Invention 7 | Ti, grade 2 | Sputtring | 170 | Good |
| Ref. 1 | Ti, grade 2 | None, Native oxide | n.a. | None |
| Ref. 2 | Ti, grade 2 | Thermal oxidation | n.a. | Poor |
| Ref. 3 | TiAl6V4 | Thermal oxidation | n.a. | None |

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

1. A method of making a coated medical bone implant, comprising the steps of:
providing a substrate, and
onto said substrate depositing a bioactive crystalline TiO₂ coating using PVD technique at a deposition temperature of more than about 50° C. but less than about 800° C.

2. A method of claim 1 wherein the PVD technique is a cathodic arc evaporation.

3. A method of claim 1 wherein the substrate, during deposition, is subjected to a 3-fold rotating motion.

4. The method of claim 1 wherein the deposited TiO₂ coating has a thickness of more than about 3 nm but less than about 5000 nm.

5. The method of claim 1 wherein the bioactive crystalline TiO₂ coating is the outermost coating.

6. The method of claim 1 further comprising preheating the substrate before deposition.

7. A coated medical bone implant comprising a substrate and a coating wherein the coating comprises a bioactive crystalline TiO₂ PVD coating.

8. A coated medical bone implant of claim 7 wherein the PVD coating is a cathodic arc evaporation coating.

9. A coated medical bone implant of claim 7 wherein the TiO₂ coating has a thickness of more than about 3 nm but less than about 5000 nm.

10. A coated medical bone implant of claim 7 wherein the bioactive crystalline TiO₂ coating is the outermost coating.
